(19) United States
(12) Patent Application Publication
(10) Pub. No.: US 2005/0232882 A1
(43) Pub. Date: Oct. 20, 2005

(54) Silicone composition-based hair fiber treating method

(76) Inventors: Cecile Bebot, Clichy (FR); Ludivine Laurent, Courbevoie (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/097,170

(22) Filed: Apr. 4, 2005

Related U.S. Application Data
(60) Provisional application No. 60/572,104, filed on May 19, 2004.

(30) Foreign Application Priority Data
Apr. 2, 2004 (FR) 04 50670

(51) Int. Cl. A61K 7/06; A61K 7/11

(52) U.S. Cl. 424/70.1; 424/70.12

(57) ABSTRACT

The present invention relates to a hair fiber treating method comprising: applying to the hair fibers a composition comprising at least 5% by weight, relative to the total weight of the composition, at least one silicone chosen from aryl silicones and silicone gums, then heating the hair fibers with a heating flat iron having a temperature ranging from 150 to 250° C.

This application claims benefit of U.S. Provisional Application No. 60/572,104, filed May 19, 2004, and French Patent Application No. 04 50670, filed Apr. 2, 2004, both of which are hereby incorporated by reference.

The present disclosure relates to a method for treating hair fibers which may be used to improve the shine of the hair fibers.

Many hair treating methods use silicone-based compounds. Silicone-based compounds are cosmetic active agents that are known to enhance the cosmetic properties of the hair. For example, they may have a conditioning effect on the hair and/or may add shine to it.

However, this shine enhancement provided by silicones often tends to fade very rapidly over time.

Many hair fiber treating methods also involve a hair drying step. For instance, patent application WO 99/17719 discloses a hair fiber treating method for conditioning and non permanent waving of the hair. This method involves applying a non rinsed composition comprising a non volatile silicone conditioning agent, a resin and a carrier base, then using a heating device to dry or to style the hair, this operation inducing a reduction of at least 1% of the bending modulus. The non volatile silicone conditioning agent represents from 0.1 to 2% by weight, relative to the total weight of the composition.

Heating the hair, however, may have a damaging effect on hair fibers.

It would thus be desirable to provide a hair fiber treating method that compensates for the drawbacks of the prior art. More particularly, it would be desirable to provide a hair fiber treating method that can add an intensive shine to hair fibers while durably maintaining this effect over time. Moreover, such a method would ideally make it possible to increase rigidity and to enhance hair fiber smoothing.

The present inventors have discovered that it is possible to counteract at least one of the above-described drawbacks of the prior art by carrying out a hair fiber treating method comprising applying to the hair fibers a composition comprising at least 5% by weight, relative to the total weight of the composition, of at least one silicone chosen from aryl silicones and silicone gums, then heating the hair fibers with a heating flat iron having a temperature ranging from 150 to 250° C.

More specifically, the present disclosure relates to a hair fiber treating method comprising:
applying onto the hair fibers a composition comprising at least 5% by weight, relative to the total weight of the composition, of at least one silicone chosen from aryl silicones and silicone gums, then
raising the temperature of the hair fibers with a heating flat iron having a temperature ranging from 150 to 250° C.

In one embodiment, the hair fibers are damp when the composition is applied.

In another embodiment, the hair fibers are pre-dried.

As described above, the at least one silicone present in the composition is chosen from aryl silicones and silicone gums.

As used herein, ‘aryl silicone’ refers to a silicone in which at least one of the repeating units comprises at least one aryl group.

Polyalkylarylsiloxanes and polydiarylsiloxanes are examples of aryl silicones suitable for use in the method disclosed herein.

Polyalkylarylsiloxanes include, for example, polyphenyltrimethyl-siloxanes (also called phenyltrimethicones) having the following formula:

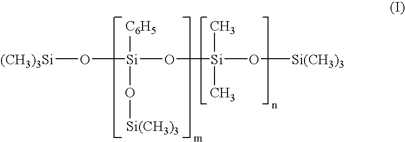

wherein:

n is an integer from 0 to 100, and

m is an integer from 1 to 400.

In one embodiment, n is zero and m is an integer from 1 to 3.

Polyphenyltrimethylsiloxanes of formula (I) wherein n is zero that are suitable for use herein include, for example, the product commercially marketed by DOW CORNING under the trade name DC 556 FLUID.

Polyphenyltrimethylsiloxanes of formula (I) wherein n differs from zero that are suitable for use herein include, for example, products commercially marketed by GOLDSCHMIDT under the trade names ABIL AV 200 and ABIL AV 1000.

Silicone gums suitable for use herein may be chosen from:
high molecular weight polydimethylsiloxanes (also called dimethicones) having the following formula:

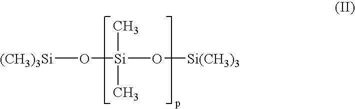

wherein p is an integer greater than 2000; such as the product commercially marketed by GENERAL ELECTRIC under the trade name VISCASIL 60M; and
polydimethylsiloxane-αω-silanols (also called dimethiconol) having the following formula:

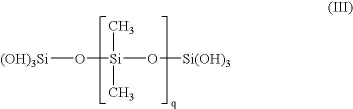

wherein q is an integer greater than 2000; such as products commercially marketed by DOW CORNING under the trade names DC SGM-3, Q2-1403, DC F2-1671 and DC 2-9071.

According to one embodiment, the composition used in the method disclosed herein comprises at least one cosmetically acceptable solvent, chosen, for example, from water; C₁-C₆ alcohols, for example, alkanols such as ethanol, propanol and isopropanol; alkanediols such as propyleneglycol, glycerol and pentanediol; benzyl alcohol; polyol ethers; C₂-C₆ esters; N-methylpyrrolidone (NMP); C₃-C₆ cetones; and silicone oils.

Silicone oils suitable for use as a solvent may be chosen, for example, from cyclomethicones having the following formula:

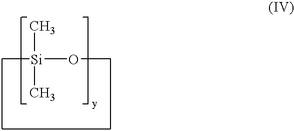

wherein y is an integer ranging from 3 to 8.

For example, cyclomethicones useful herein include cyclotetradimethylsiloxane (y=4), cyclopentadimethylsiloxane (y=5) and cyclohexadimethylsiloxane (y=6).

Products commercially marketed by DOW CORNING under the trade names DC FLUID 244, DC FLUID 245, DC FLUID 344 and DC FLUID 345 may be used, for example.

In one embodiment, the composition applied according to the method disclosed herein is anhydrous.

The solvent employed is, in one embodiment, ethanol or a silicone oil.

In order to improve the cosmetic properties of the hair fibers or to prevent them from being damaged or to reduce their damaging, the composition disclosed herein may also comprise at least one additional cosmetic active agent.

The at least one cosmetic active agent may, for instance, be chosen from organomodified silicones such as amine group containing silicones, cationic, anionic, amphoteric or non ionic polymers, peptides and derivatives thereof, protein hydrolyzates, waxes, swelling agents and penetrating agents such as dimethylisosorbitol, urea and derivatives thereof, anionic, cationic, non ionic, amphoteric or zwitterionic surfactants, active agents for combating hair loss, anti-dandruff agents, natural or synthetic, associative or non-associative thickeners, suspension agents, sequestering agents, opacifying agents, dyes, sunscreen agents, vitamins and provitamins, fatty acids, fatty alcohols, mineral, vegetable or synthetic oils, as well as fragrances and preserving agents, and any combination thereof.

The at least one additional cosmetic active agent may be present in an amount ranging from 0.01 to 30%, such as from 0.1 to 10% by weight, relative to the total weight of the composition.

The pH of the composition disclosed herein ranges from 2 to 13, such as from 4 to 10.

The pH of the composition may be adjusted by an alkaline agent, such as, for example, ammonia, monoethanolamine, diethanolamine, triethanolamine, 1,3-propanediamine, alkaline or ammonium carbonate or bicarbonate, organic carbonate such as guanidine carbonate, alkaline hydroxide or by an acidifying agent such as, for example, hydrochloric acid, acetic acid, lactic acid, oxalic acid or boric acid.

The composition used in the method disclosed herein may come in the form of a lotion, thickened or not, a cream, a gel or a foam.

In one embodiment, the composition is not in aerosol form.

The composition may be applied to the hair fibers in an amount ranging from 0.05 to 0.3 g, such as from 0.1 to 0.2 g, of the composition per gram of dry hair fiber.

After the composition has been applied to the hair fiber, and before the hair fiber is heated with a heating flat iron, the composition may be left on for a period of time ranging from 30 seconds to 60 minutes, such as from 5 to 45 minutes.

As explained above, in the method according to the present disclosure, after the composition is applied to the hair fibers, the hair fibers are heated with a heating flat iron having a temperature ranging from 150° C. to 250° C.

As used herein, ‘iron’ refers to a hair fiber heating device which functions by contacting the hair fibers with the heating device.

The end of the iron which comes into contact with the hair has two plane surfaces. These two plane surfaces may be made of metal. They can be smooth or notched.

All types of flat irons may be given as suitable irons for use in the method disclosed herein; as a non-limiting example, those described in U.S. Pat. No. 5,957,140 and No. 5,046,516, may be used.

The iron may be applied proceeding by successive separated touches over about a few seconds or by gradually moving or sliding along hair sections.

In one embodiment, the iron is applied, once or several times, using a continuous gliding movement from the hair roots to the ends.

The process according to the present disclosure may also comprise an additional step comprising partially pre-drying hair fibers before heating, so as to prevent any substantial steam development that might bum the hands of the hair stylist and the scalp of the user. This pre-drying step may be done, for example, by using a hair drier, a hood, or it is also possible to let the hair dry naturally.

As explained above, the method according to the present disclosure may contribute to improvement of the shine intensity while durably prolonging this effect over time.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements.

The following example is intended to illustrate the invention in a non-limiting manner.

### EXAMPLE

The hair fiber treating method as disclosed herein was carried out using a composition comprising a silicone compound.

The tested compositions were as follows:

### Composition 1

| | |
|---|---|
| DC 2-9071 | 7% AM |
| Ethanol | 5% |
| Fragrances and preserving agents | 0.8% |
| DC 245 FLUID | QS 100 |

AM: active material

### Composition 2

| | |
|---|---|
| DC 556 FLUID | 7% |
| Fragrance | 0.4% |
| Ethanol | QS 100 |

The hair treatment was carried out as follows:
the composition was applied onto damp hair, in an amount of 0.15 g per gram of dry hair;
the hair was pre-dried with a hair-dryer until the hair was nearly drd (pre-drying to 80%);
a flat iron (iron commercially marketed under the trade name Techniliss loni Ceramic # PNC 228 by VELECTA), was applied to the hair in a continuous movement from the hair roots to the ends, once or several times; the temperature of the iron being about 210° C.

As a result, the hair showed an intensive and long-lasting shine.

1. A method for treating hair fibers, comprising:
applying to said hair fibers a composition comprising at least 5% by weight, relative to the total weight of the composition, of at least one silicone chosen from aryl silicones and silicone gums, then
heating the hair fibers with a heating flat iron having a temperature ranging from 150 to 250° C.

2. A method according to claim 1, wherein the composition is applied onto damp hair fibers.

3. A method according claim 1, wherein the aryl silicones are chosen from polyalkylarylsiloxanes and polydiarylsiloxanes.

4. A method according to claim 3, wherein the polyalkylarylsiloxanes are chosen from polyphenyltrimethylsiloxanes having formula: 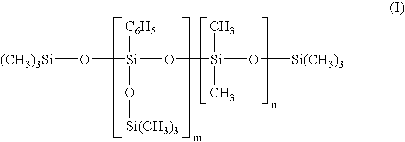
wherein:
n is an integer from 0 to 100, and
m is an integer from 1 to 400.

5. A method according to claim 4, wherein in formula (I) n is zero and m is an integer from 1 to 3.

6. A method according to claim 1, wherein the silicone gums are chosen from:
polydimethylsiloxanes having formula: 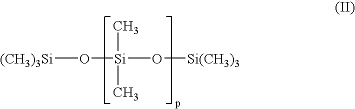
wherein p is an integer greater than 2000; and
polydimethylsiloxane-α,ω-silanoIs having formula: 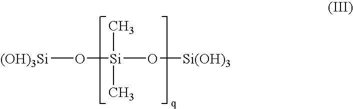
wherein q is an integer greater than 2000.

7. A method according to claim 1, wherein the composition further comprises at least one cosmetically acceptable solvent chosen from water, C₁-C₆ alcohols, alkanediols, benzyl alcohol, polyol ethers, C₂-C₆ esters, N-methylpyrrolidone (NMP), C₃-C₆ cetones and silicone oils.

8. A method according to claim 7, wherein said C₁-C₆ alcohols are alkanols chosen from ethanol, propanol and isopropanol.

9. A method according to claim 7, wherein said alkanediols are chosen from propyleneglycol, glycerol and pentanediol.

10. A method according to claim 1, wherein said composition is anhydrous.

11. A method according to claim 1, wherein said composition also comprises at least one additional cosmetic active agent chosen from organomodified silicones, cationic, anionic, amphoteric and non ionic polymers, peptides and derivatives thereof, protein hydrolyzates, waxes, swelling agents and penetrating agents, anionic, cationic, non ionic, amphoteric and zwitterionic surfactants, active agents for combating hair loss, anti-dandruff agents, natural or synthetic, associative and non-associative thickeners, suspension agents, sequestering agents, opacifying agents, dyes, sunscreen agents, vitamins and provitamins, fatty acids, fatty alcohols, mineral, vegetal and synthetic oils, fragrances and preserving agents, and mixtures thereof.

12. A method according to claim 11, wherein the at least one additional cosmetic active agent is present in an amount ranging from 0.01 to 30% by weight, relative to the total weight of the composition.

13. A method according to claim 12, wherein the at least one additional cosmetic active agent is present in an amount ranging from 0.1 to 10% by weight, relative to the total weight of the composition.

14. A method according to claim 1, wherein said composition has a pH ranging from 2 to 13.

15. A method according to claim 14, wherein said composition has a pH ranging from 4 to 10.

16. A method according to claim 1, wherein said composition is in the form of an optionally thickened lotion, a cream, a gel or a foam, but wherein said composition is not in the form of an aerosol.

17. A method according to claim 1, comprising, after applying the composition to the hair fibers and before heating the hair with the heating flat iron, leaving the composition on the hair fibers for a period of time ranging from 30 seconds to 60 minutes.

18. A method according to claim 1, wherein, after applying the composition to the hair fibers and before heating the hair with the heating flat iron, partially pre-drying the hair fibers.

19. A method for providing improved and prolonged shine intensity of hair fibers comprising:
applying to said hair fibers a composition comprising at least 5% by weight, relative to the total weight of the composition, of at least one silicone chosen from aryl silicones and silicone gums, then
heating the hair fibers with a heating flat iron having a temperature ranging from 150 to 250° C.
